(19) United States
(12) Patent Application Publication
(10) Pub. No.: US 2006/0045845 A1
(43) Pub. Date: Mar. 2, 2006

(54) NeuX- a new composition of matter

(76) Inventor: Willard H. Schmidt, Albuquerque, NM (US)

Correspondence Address: Willard H. Schmidt, 2015 Altez NE, Albuquerque, NM, 87112, US

(21) Appl. No.: 10/893,394

(22) Filed: Jul. 19, 2004

(51) Int. Cl. A61K 51/00

(52) U.S. Cl. 424/1.11

(57) ABSTRACT

This patent application is for a new composition of matter called “neuX”. It consists of two or more fundamental particles, called neutrons, that have no electric charge and are bound together by the so-called “strong forces”. Units of neuX, which may consist of any number of neutrons, are for use in the creation of nuclear medicines and for therapeutic treatment to help cure all types of cancer, cancer bone pain, hyperthyroidism, polycythaemia, arthritis, and many other diseases. Units of neuX can also be used in the creation of many hydrogenous compounds, and other chemical compounds that have a variety of medical and industrial applications. NeuX units are formed of neutrons obtained from nuclear reactors, and also by many different nuclear reactions including the bombardment of certain elements, such as beryllium, with alpha particles from radioactive sources. Neutrons can be released by the irradiation of deuterium with high-energy gamma rays. It is well known that free neutrons are radioactive with a half-life of about fifteen minutes. However, in a strongly bound state, such as in a neuX unit or the deuteron, which consists of a neutron and a proton, there is no radioactivity.

### BACKGROUND

1. Field of Invention

This invention is for a new composition of matter called “neuX”. It consists of units made up of any number of neutrons that are bound together by the so-called strong nuclear force. Neutrons are electrically neutral fundamental particles that are classified as Baryons. Units of neuX are for use in the creation of nuclear medicines, therapeutic treatments, and certain industrial applications. In the name “neuX”, the X refers to the number of neutrons in the unit. For example, neu2, indicates that there two neutrons in the neuX unit.

The atomic nucleus of every element consists of positively charged protons and electrically neutral neutrons in numbers that describe that particular element. Orbital electrons with a negative electrical charge balance the positive electrical charge of a nucleus. The addition of extra neutrons to a nucleus creates what is known as an isotope. A unit of neuX is not an atom or an isotope since it is made up only of neutrons and contains no positively charged protons.

2. Description of Prior Art

Nuclear medicines commonly use substances that are radioactive. Radiation is obtained from radioactive isotopes and is directed toward a diseased area in a patient in order to treat the disease. Radioisotopes are obtained from the fission products of nuclear reactors and are also created by neutron bombardment of certain target materials in a nuclear complex designed for that purpose.

Nuclear medicine also includes high energy photon or ganuna irradiation of diseased regions in a patient. This requires the use of very expensive high-tech electron accelerator equipment to create the desired radiation.

Objects adn Advantages

This invention is for a new composition of matter, called neuX, for which there are many medical and industrial uses. In common practice the creation of radioactive isotopes for both medical and industrial applications is highly technical, and hazardous from both a toxicity standpoint and radiation exposure. Large nuclear facilities with highly trained personnel are required. The use of neuX units simplifies the operations of creating new radioactive isotopes, reduces occupational hazards, and minimizes cost. Units of neuX can also be used directly in medical and industrial applications without the use of isotopes.

### DRAWING FIGURES

FIG. 1 and FIG. 2 illustrate two examples of neuX units. They are called “neu2” and “neu4” since they consist of two and four neutrons respectively. In the figures the neutrons are labeled number 1, and double-headed arrows in the figures indicate the strong forces binding the neutrons together in the neuX units.

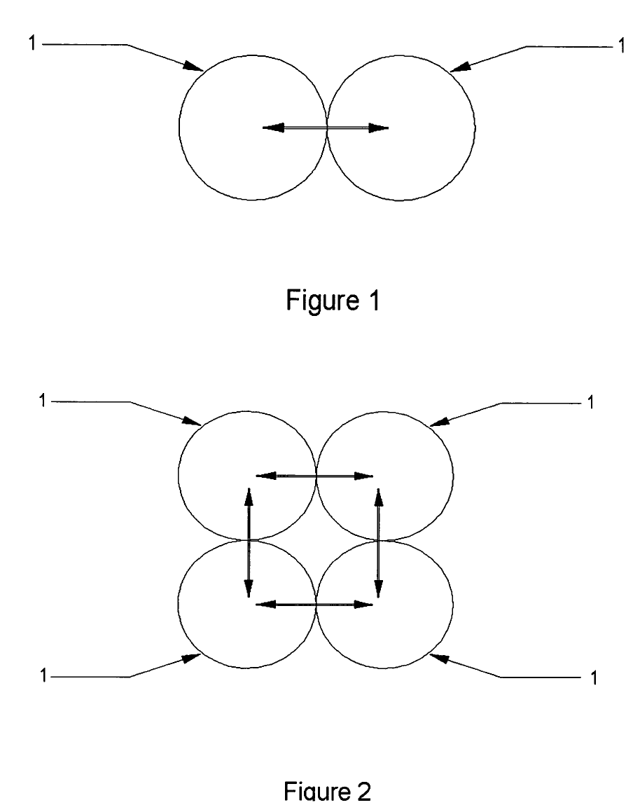

Operational Usage

With the use of special equipment and controls, neuX units are formed from free neutrons, as indicated above. Then, in a special process, neuX units are co-joined to selected atomic nuclei to create specific isotopic radioactive source nuclei. For example, radioactive strontium-90 can be made by co-joining stable strontium-88 with neu2. Radioactive cerium-144 can be produced by co-joining stable cerium-140 with neu4 units. Bismuth-213 is a strong radioactive isotopic radiation source and can be made by co-joining stable bismuth-209 with neu4. Again, with special equipment designed for the purpose, units of neuX can be directed to either elemental targets or to diseased regions in human patients for therapeutic treatments.

I claim:

1. A new composition of matter, called neuX, for use in many medical and industrial applications.

2. The neuX matter in claim 1 is made up of fundamental neutron particles, classified as hadrons, that are held together by the so-called strong nuclear forces.

3. The neuX matter in claim 1 can contain any number of neutrons.

4. Units of neuX matter can be directed at elemental targets to create radioactive isotopes whose radiation can be used for both medical and industrial purposes.

5. Units of neuX matter can be directed at diseased regions in human patients for therapeutic treatments and cure of diseases.
